(19) United States
(12) Patent Application Publication
(10) Pub. No.: US 2018/0368708 A1
(43) Pub. Date: Dec. 27, 2018

(54) Photoplethysmogram Signal Measurement Device for Exercise Equipment

(71) Applicant: Bion Inc., New Taipei City (TW)

(72) Inventor: Yu-Yu CHEN, Taipei City (TW)

(21) Appl. No.: 15/630,930

(22) Filed: Jun. 22, 2017

(51) Int. Cl. A61B 5/024; A61B 5/00

(52) U.S. Cl. CPC A61B 5/02427; A61B 5/6895

(57) ABSTRACT

A photoplethysmogram signal measurement device for exercise equipment is provided, which includes a photoplethysmogram signal detector for detecting a volumetric variation caused by a blood pressure pulse of a user through a skin surface of a user and then a series of heart rate signals are generated. The photoplethysmogram signal detector includes a carrier, a light emitting device, a light receiving device, a signal digitizer, a processing unit and a detection actuating unit. The detection actuating unit is used to generate an enabling signal to actuate the light receiving device to start receiving a series of photoplethysmogram signals and actuate the processing unit to start receiving a digitized photoplethysmogram signals from the signal digitizer. An optical grating is disposed between the light emitting device and the light receiving device for blocking an outside interference light from interfering the light receiving device.

### BACKGROUND OF THE INVENTION

### 1. Field of the Invention

The present invention relates to a personal exercise measuring system, and more particularly, to an exercise measurement system using photoplethysmogram measurement technique for exercise equipment to detect exerciser's heart rate during exercise.

### 2. Description of the Prior Art

Riding a bicycle has become a trend for many reasons such as sport and travel. It is often that exerciser engaged in outdoor sports tends to focus on sports and ignore their own physiological conditions. If no proper measurement or evaluation is taken to monitor the health condition of the rider, the rider could be harmed from excessive bicycle riding.

Therefore, it is necessary to provide an exercise measuring system which can monitor the health condition of the rider. Various health monitoring devices are developed in prior arts.

Electrocardiography (EKG) is one of prior arts, capable of recording the electrical activity of the heart over a period of time using electrodes placed on the skin of the user. These electrodes detect the tiny electrical changes on the skin that arise from the heart muscle's electrophysiologic pattern of depolarizing and repolarizing during each heartbeat. It is a very commonly performed cardiology test.

The EKG electrodes may be installed on a handle of an exercise equipment such as indoor fitness equipment, outdoor bike or scooter, serving as a heartbeat detection device for detecting exercising status of a user during exercise. However, EKG electrodes are generally installed on two separate handles of the exercise equipment to measure the user's heartbeat. So, the user must hold his two hands on the handles in order to accurately measure the heartbeat signals. Otherwise, it is easy to lead to an inaccurate measurement. In addition, it is often that the EKG circuit receives unwanted signal noise due to the user's hand posture, hand sweat, or hand dirt during exercise. In order to overcome this problem, a compensation circuit is additionally required in the EKG circuit.

Another approach used to monitor the user's heart rate during exercise in prior arts is using optical components. Although the technique of measuring and calculating the heartbeat signals of the user by using the optical method is basically feasible, it can not accurately measure the heartbeat signals for various factors such as vibration, and outside interference light.

### SUMMARY OF THE INVENTION

To solve the previous technical problems, one objective of the present application is providing a exercise measuring system which uses photoplethysmogram measurement technique to monitor exerciser's heart rate during exercise.

To achieve the aforementioned objective, the present application provides a photoplethysmogram signal measurement device for exercise equipment, which includes a photoplethysmogram signal detector for detecting a volumetric variation caused by a blood pressure pulse of a user through a skin surface of a user and then a series of heart rate signals are generated. The photoplethysmogram signal detector includes a carrier, a light emitting device, a light receiving device, a signal digitizer, a processing unit and a detection actuating unit. The detection actuating unit is used to generate an enabling signal to actuate the light receiving device to start receiving a series of photoplethysmogram signals and actuate the processing unit to start receiving digitized photoplethysmogram signals from the signal digitizer. An optical grating is disposed between the light emitting device and the light receiving device for blocking an outside interference light from interfering the light receiving device.

The photoplethysmogram signal detector is design to face an acupuncture point on the palm of the user in order to accurately measure the user's heartbeat signal.

In the aspect of sports ergonomics, the user can grasp the grip by his five fingers and grasp the grip in a palm to achieve the purpose of measuring the heartbeat signals with safe and comfortable exercise.

The present invention overcomes the problem that the user must hold his two hands on the handles of the traditional EKG technology, without need of signal compensation circuit.

In the design of the present invention, a grating is arranged the light emitting device and the light receiving device, so that a largest light shielding area is obtained and a better interference from an outside light is effectively achieved when the grip of the photoplethysmogram signal measurement device is contacted with the user's skin.

### BRIEF DESCRIPTION OF THE DRAWINGS

For a better understanding of the aforementioned embodiments of the invention as well as additional embodiments thereof, reference should be made to the Description of Embodiments below, in conjunction with the following drawings in which like reference numerals refer to corresponding parts throughout the figures.

FIG. 1 is a perspective view showing a photoplethysmogram signal measurement device in accordance with a preferred embodiment of the present invention;

FIG. 2 is an exploded view showing the photoplethysmogram signal measurement device of FIG. 1.

FIG. 3 is a cross-sectional view taken along lines 3-3 of FIG. 1;

FIG. 4 is a cross-sectional view taken along lines 4-4 of FIG. 1;

FIG. 5 is a cross-sectional view showing that a resistive detection actuating unit is formed on an outer surface of grip;

FIG. 6 is a functional circuit diagram in accordance with a first embodiment of the present invention;

FIG. 7 illustrates a functional circuit diagram in accordance with a second embodiment of the present invention;

FIG. 8 is a view showing the photoplethysmogram signal measurement device of the present invention is handheld by a palm of a user during exercise;

FIG. 9 is a view showing the photoplethysmogram signal measurement device of the present invention is installed on a steering wheel of a car;

FIG. 10 is a view showing the photoplethysmogram signal measurement device of the present invention is installed on an indoor fitness equipment;

FIG. 11 is a view showing the photoplethysmogram signal measurement device of the present invention is installed on an outdoor bike;

FIG. 12 is a functional circuit diagram in accordance with a third embodiment of the present invention;

FIG. 13 is a functional circuit diagram in accordance with a fourth embodiment of the present invention;

FIG. 14 is a functional circuit diagram in accordance with a fifth embodiment of the present invention; and

FIG. 15 illustrates a functional circuit diagram in accordance with a sixth embodiment of the present invention.

### DETAILED DESCRIPTION OF THE PREFERRED EMBODIMENT

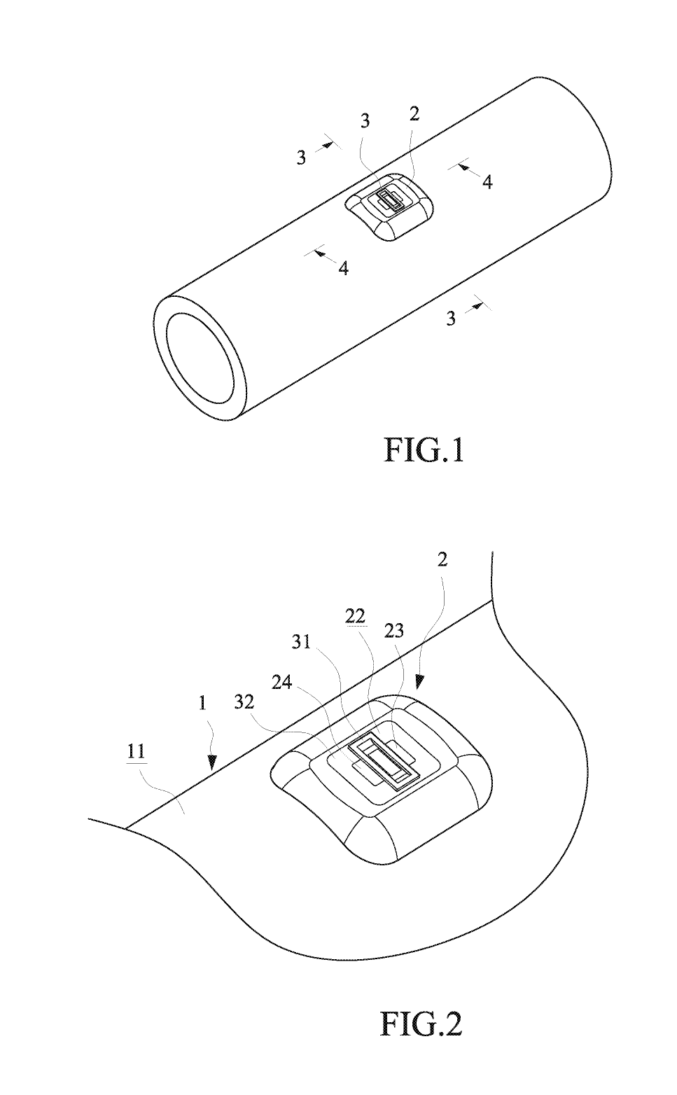

With reference to FIGS. 1 and 2, FIG. 1 is a perspective view showing a photoplethysmogram signal measurement device in accordance with a preferred embodiment of the present invention, and FIG. 2 is an exploded view showing the photoplethysmogram signal measurement device of FIG. 1. As shown in the drawings, the photoplethysmogram signal measurement device comprises a grip 1 adapted to be gripped by a palm of a user. The grip 1 has an outer surface 11 and an inner surface 12.

In other embodiment of the present invention, the grip 1 may be in a form of a wearable electronic device suitable to be wore on a selected portion, such as wrist, of the user.

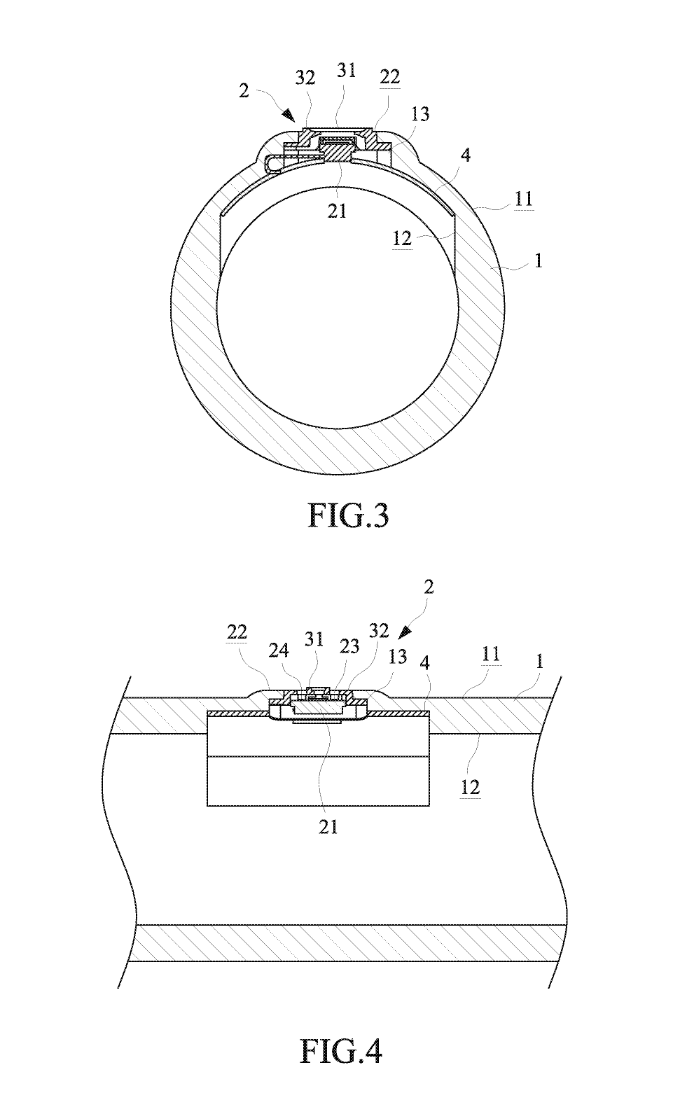

Referring also to FIGS. 3 and 4, in which FIG. 3 is a cross-sectional view taken along lines 3-3 of FIG. 1, and FIG. 4 is a cross-sectional view taken along lines 4-4 of FIG. 1.

A photoplethysmogram signal detector 2 is mounted in an excavated area 13 of the grip 1. Preferably, the photoplethysmogram signal detector 2 is arranged to face an acupuncture point on the palm of the user in order to accurately measure the user's heartbeat signal.

The photoplethysmogram signal detector 2 includes a carrier 21 which is fitted in the excavated area 13 of the grip 1. The carrier 21 defines a skin contact surface 22.

The photoplethysmogram signal detector 2 includes a light emitting device 23 and a light receiving device 24. The light emitting device 23 is disposed on the carrier 21, capable of generating an optical signal L1 outward from the skin contact surface 22 of the carrier 21. The light receiving device 24 is also disposed on the carrier 21 corresponding to the light emitting device 23 with a distance, adapted to receive the optical signal L1 generated by the light emitting device 23.

An optical grating 31 is disposed on the carrier 21 and positioned between the light emitting device 23 and the light receiving device 24. The optical grating 31 is preferably disposed on the carrier 21 with a slight protrusion with respect to the contact surface 22 of the carrier 21. The optical grating 31 may further comprise a surrounding portion 32 which surrounds the light emitting device 23 and the light receiving device 24.

A detection actuating unit 4 is mounted under the carrier 21 and arranged on the inner surface 12 of the grip 1. The detection actuating unit 4 is in a form of plate form, which constitutes a capacitor sensing device with the grip 1 made of insulation material, for detecting the capacitive variation during the skin contact surface 22 of the carrier 21 is touched by a user.

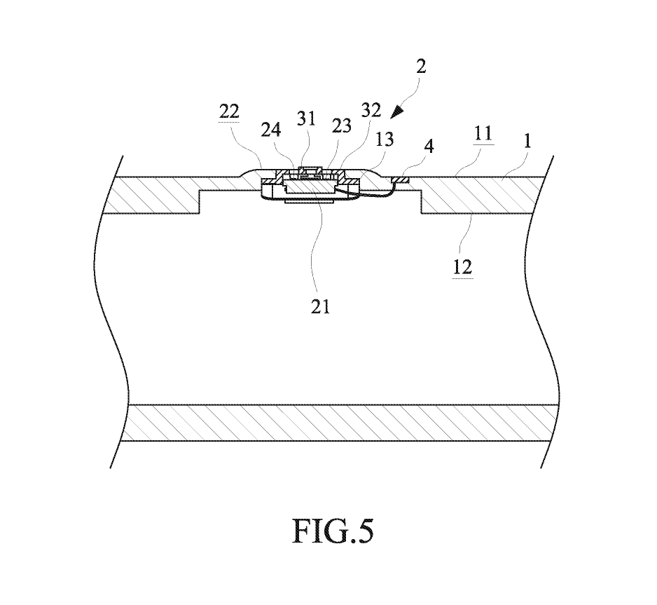

As shown in FIG. 5, the detection actuating unit 4 may be in a form of contactable plate formed on the outer surface 11 of grip 1 for detecting the resistive variation during the skin contact surface 22 of the carrier 21 is touched by a user. Alternatively, the detection actuating unit 4 may be formed on the skin contact surface 22 of the carrier 21.

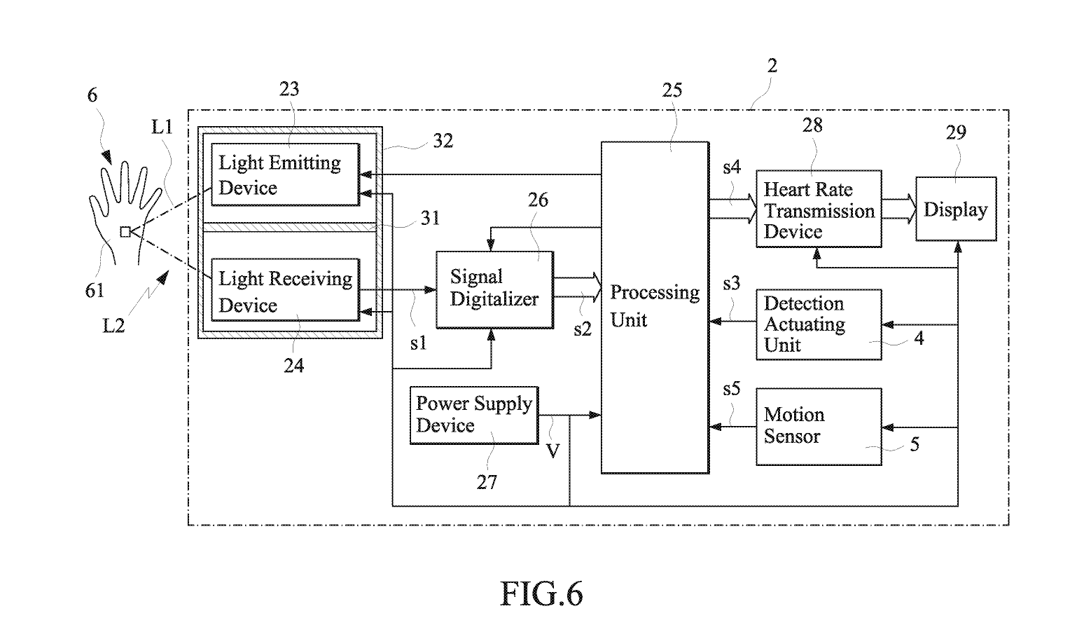

FIG. 6 illustrates a functional circuit diagram in accordance with a first embodiment of the present invention. As shown in the drawing, the light emitting device 23 is electrically connected to a processing unit 25. The light receiving device 24 is used to receive the optical signal L1 generated by the light emitting device 23 and then generating a series of photoplethysmogram signals s1.

A signal digitizer 26 is connected to the light receiving device 24 for receiving and converting the series of photoplethysmogram signals s1 into digitized photoplethysmogram signals s2, and then the digitized photoplethysmogram signals s2 are sent to the processing unit 25.

An power supply device 27 is used to supply an operating voltage V to the light emitting device 23, the light receiving device 24, the processing unit 25, the signal digitizer 26, a heart rate signal transmission device 28, a display 29 and the detection actuating unit 4. The power supply device 27 may be either an internal power supply unit which is built in the photoplethysmogram signal detector 2 or an external power supply device which supplies the operating voltage V to the photoplethysmogram signal detector 2 through a known connector.

The detection actuating unit 4 is electrically connected to the processing unit 25 for generating an enabling signal s3 to the processing unit 25 to actuate the light receiving device 24 to start receiving the optical signal L1 of the light emitting device 23 and actuate the processing unit 25 to start receiving the series of photoplethysmogram signals s1 of the light receiving device 24.

The detection actuating unit 4 and the optical grating 31 in combination serve as an interference preventing device for preventing the abnormal operation of the photoplethysmogram signal detector 2 caused by the outside interference light L2 projected to the light receiving device 24.

The photoplethysmogram signal detector 2 may further comprise a motion sensor 5. The motion sensor 5 may be a vibration detector or an accelerometer. The motion sensor 5 is electrically connected to the processing unit 25 for generating a motion detection signal s5 to the processing unit 25 when detecting a vibration or acceleration of the photoplethysmogram signal detector 2.

When the skin contact surface 22 of the carrier 21 is touched by a skin surface 61 of a user 6, an enabling signal s3 is transmitted to the processing unit 25. At this time, the light emitting device 23 illuminates the skin surface 61 of the user 6, so that the light receiving device 24 measures a volumetric variation caused by a blood pressure pulse on the palm of the user 6 through the skin surface 61 of the user 6. Thereafter, a series of photoplethysmogram signals s1 are generated by the light receiving device 24, a series of digitized photoplethysmogram signals s2 are transmitted to the processing unit 25, and then a series of heart rate signals s4 in correspondence to the digitized photoplethysmogram signals s2 are generated by the processing unit 25. Finally, the heart rate signals s4 are transmitted through the heart rate signal transmission device 28 to the display 29 for display.

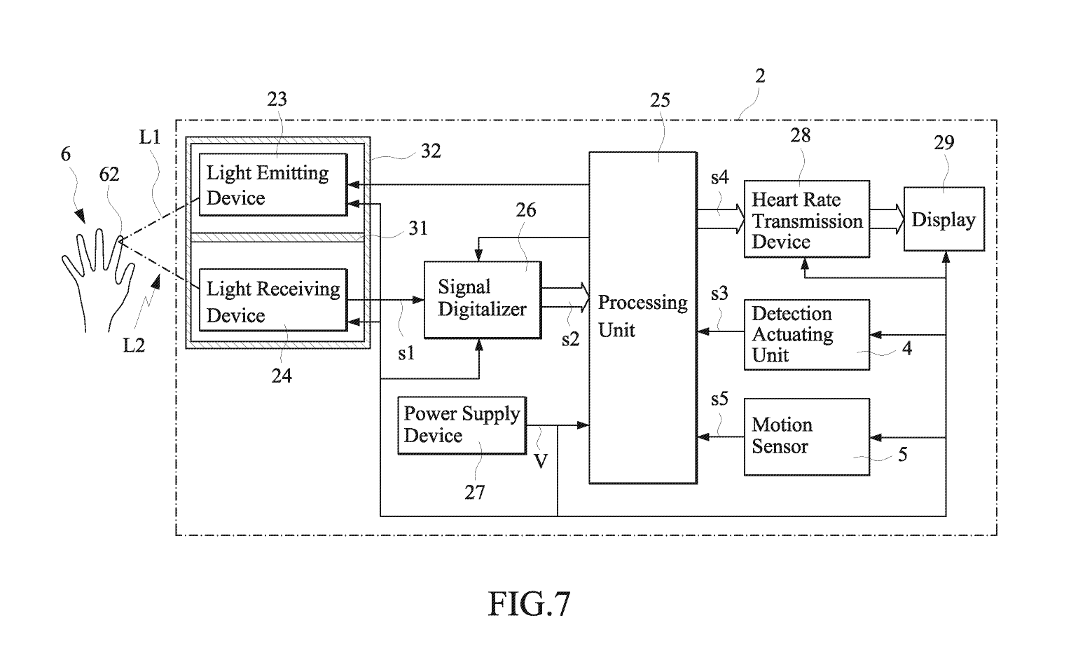

FIG. 7 illustrates a functional circuit diagram in accordance with a second embodiment of the present invention. The circuit diagram of this embodiment is identical to that of the embodiment shown in FIG. 6. It shows that the light emitting device 23 also applies to illuminate the skin surface of a finger 62 of the user 6, so that the light receiving device 24 measures a volumetric variation caused by a blood pressure pulse on the finger 62 to generate the photoplethysmogram signals s1.

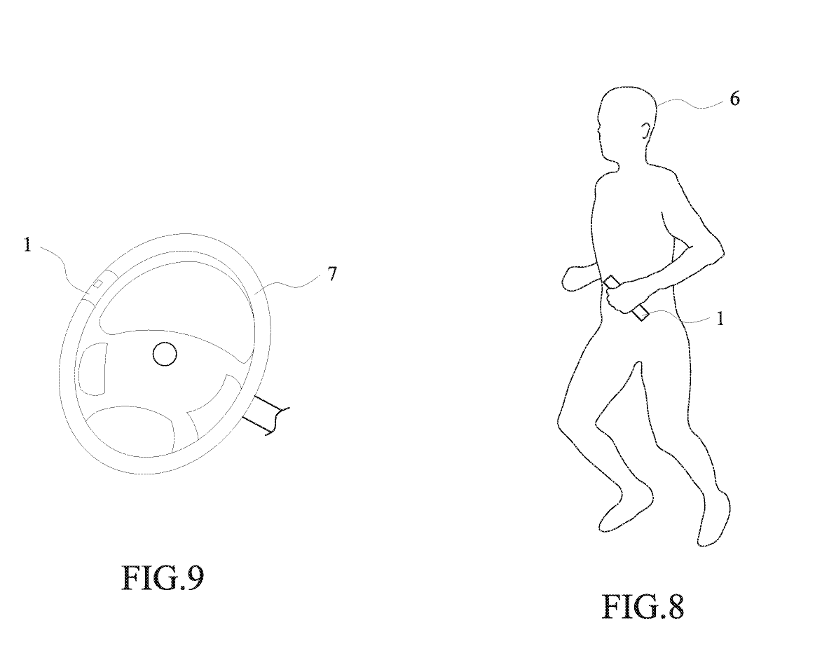

With reference to FIG. 8, it is a view showing the photoplethysmogram signal measurement device of the present invention is handheld by a palm of a user 6 during exercise. FIG. 9 is a view showing the photoplethysmogram signal measurement device 1 of the present invention is installed on a steering wheel 7 of a car.

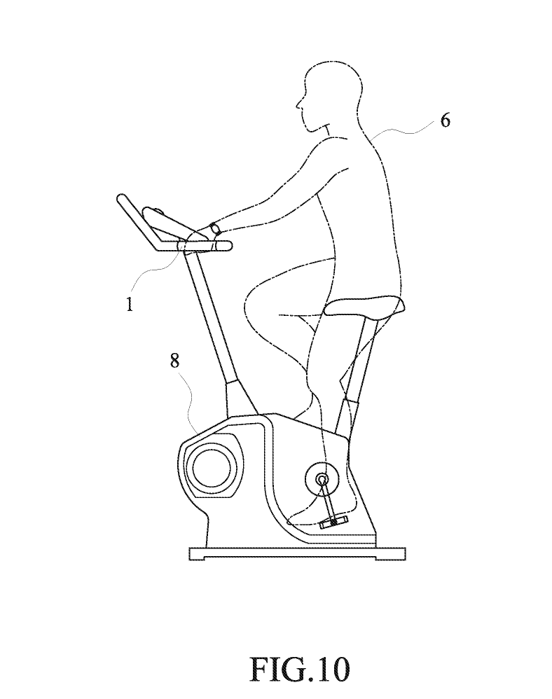

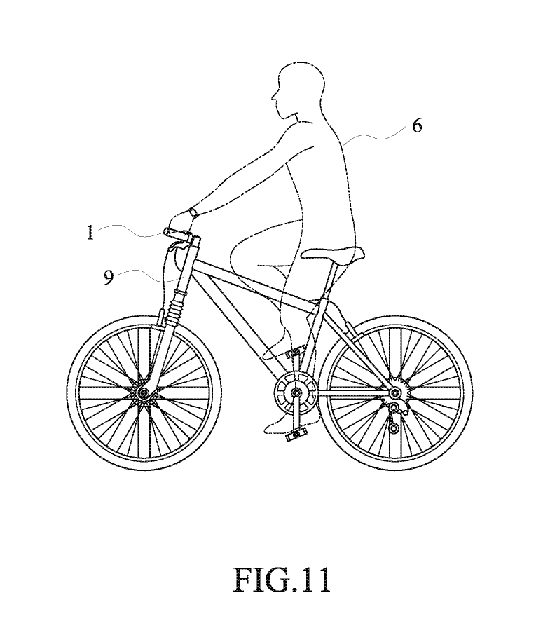

FIG. 10 is a view showing the photoplethysmogram signal measurement device 1 of the present invention is installed on an indoor fitness equipment 8. FIG. 11 is a view showing the photoplethysmogram signal measurement device 1 of the present invention is installed on an outdoor bike 9.

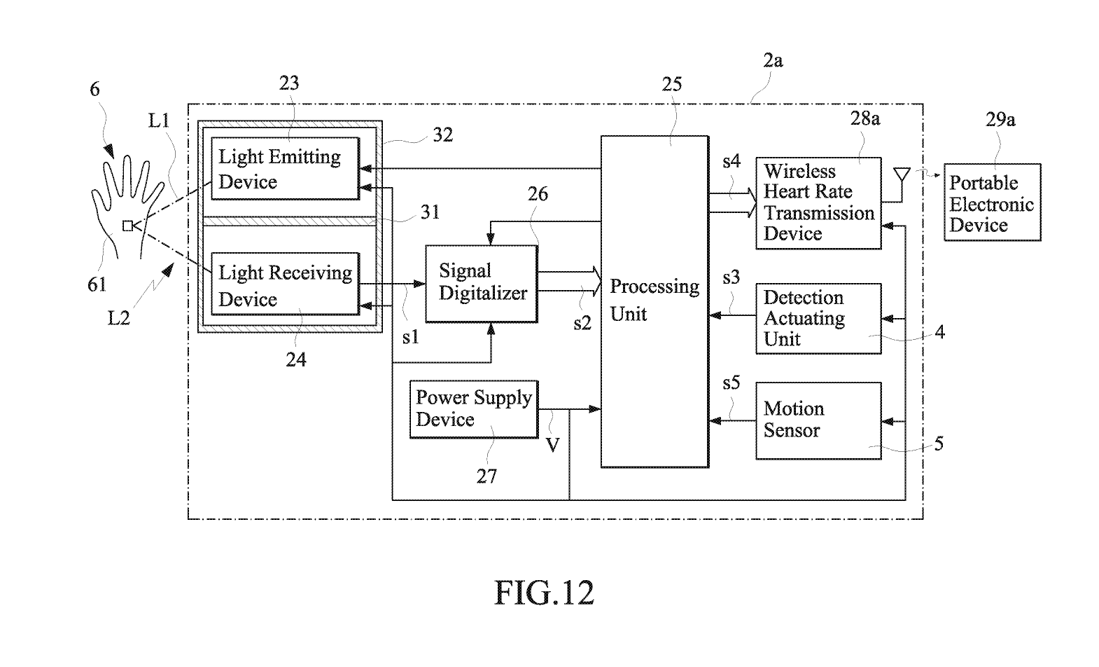

FIG. 12 illustrates a functional circuit diagram in accordance with a third embodiment of the present invention. The photoplethysmogram signal detector 2a of the second embodiment is different from the first embodiment of FIG. 6 in that the display 29 of the first embodiment is replaced with a portable electronic device 29a. The portable electronic device 29a may be a mobile phone or a personal digital assistant. Further, the heart rate signals s4 are transmitted through a wireless heart rate signal transmission device 28a to the portable electronic device 29a.

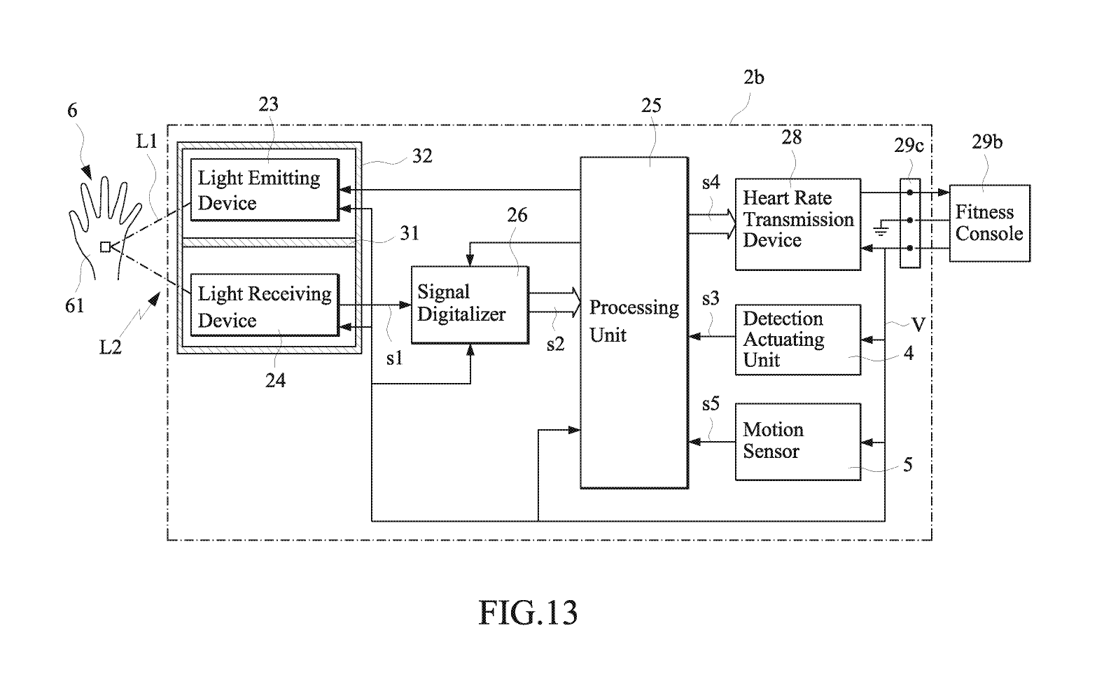

FIG. 13 illustrates a functional circuit diagram in accordance with a fourth embodiment of the present invention. The photoplethysmogram signal detector 2b of the third embodiment is different from the first embodiment of FIG. 6 in that the heart rate signal transmission device 28 of the first embodiment is connected to a fitness console 29b of an indoor fitness equipment, an outdoor bike or a scooter through a connector 29c. In this instant, the heart rate signals s4 sent from the processing unit 25 are transmitted through the heart rate signal transmission device 28 and the connector 29c to the fitness console 29b for display. Further, an operating voltage V is supplied from the fitness console 29b to the photoplethysmogram signal detector 2 through the connector 29c.

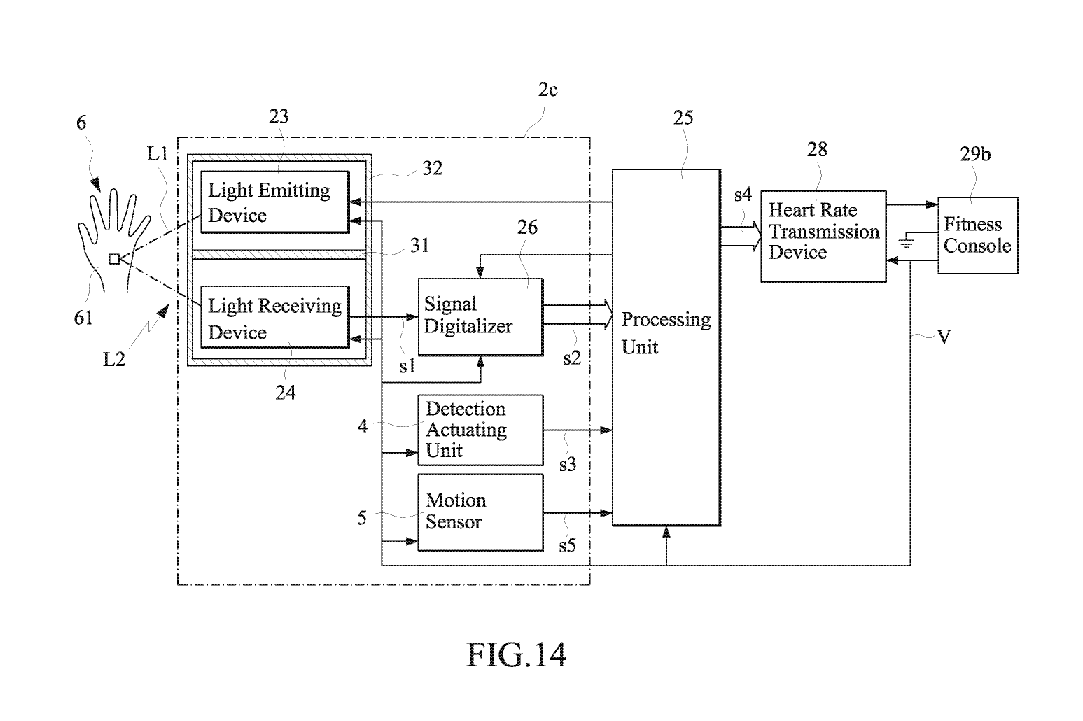

FIG. 14 is a functional circuit diagram in accordance with a fifth embodiment of the present invention. The instant embodiment comprises components/parts that are generally similar to those of the third embodiment shown in FIG. 13 and similar components/parts are designated with the same reference numerals for consistency. In this embodiment, the light emitting device 23, the light receiving device 24, the signal digitizer 26, the detection actuating unit 4, the motion sensor 5, the optical grating 31, and the surrounding portion 32 are installed in the photoplethysmogram signal detector 2c of the fifth embodiment, while the processing unit 25 and the heart rate signal transmission device 28 are included in the fitness console 29b.

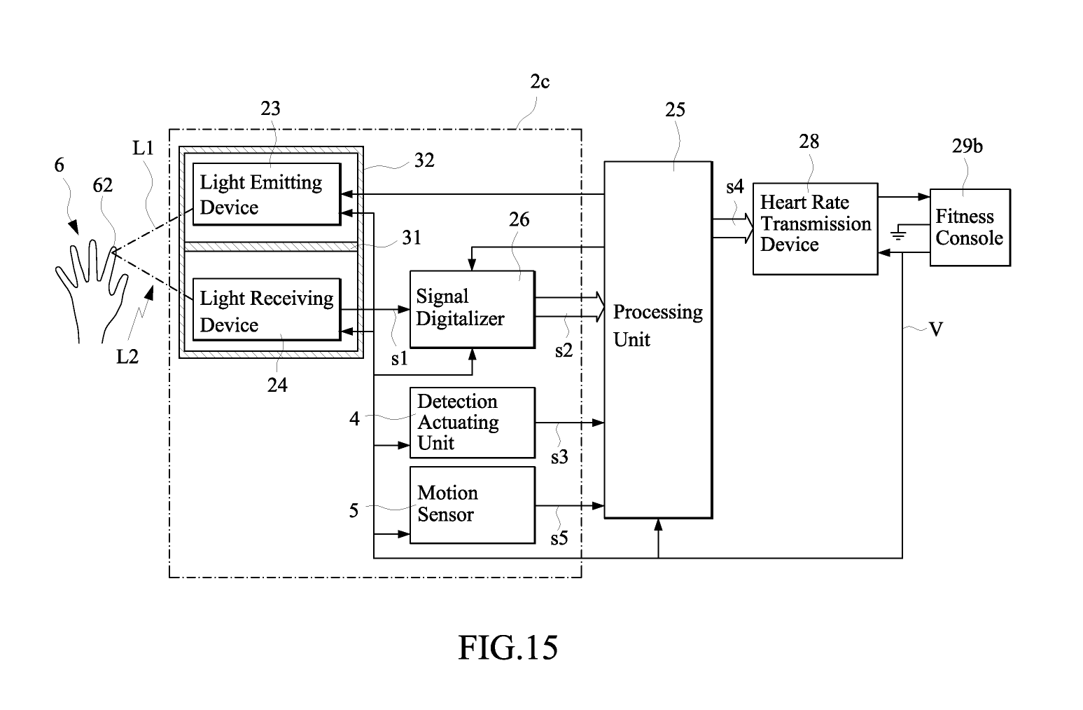

FIG. 15 illustrates a functional circuit diagram in accordance with a sixth embodiment of the present invention. The circuit diagram of this embodiment is identical to that of the embodiment shown in FIG. 14. It further shows that the light emitting device 23 also applies to illuminate the skin surface of a finger 62 of the user 6, so that the light receiving device 24 measures a volumetric variation caused by a blood pressure pulse on the finger 62 to generate the photoplethysmogram signals s1.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A photoplethysmogram signal measurement device for an exercise equipment, comprising:
a grip formed with an excavated area;
a photoplethysmogram signal detector, including:
a carrier mounted in the excavated area of the grip, the carrier defining a skin contact surface;
a light emitting device disposed on the carrier for generating an optical signal;
a light receiving device disposed on the carrier and corresponding to the light emitting device with a distance for receiving the optical signal generated by the light emitting device and then generating a series of photoplethysmogram signals;
a signal digitizer electrically connected to the light receiving device for receiving and converting the series of photoplethysmogram signals into a series of digitized photoplethysmogram signals; and
a processing unit electrically connected to the light emitting device and the signal digitizer for receiving the digitized photoplethysmogram signals from the signal digitizer;
a power supply device for supplying an operating voltage to the photoplethysmogram signal detector;
a detection actuating unit electrically connected to the processing unit for generating an enabling signal to the processing unit to actuate the light receiving device to start receiving the series of photoplethysmogram signals and actuate the processing unit 25 to start receiving the digitized photoplethysmogram signals;
an optical grating disposed on the carrier for blocking an outside interference light from interfering the light receiving device; and
a heart rate signal transmission device electrically connected to the processing unit;
wherein when the skin contact surface of the carrier is touched by a skin surface of a user thereby an enabling signal is sent to the processing unit, the light emitting device illuminates the skin surface of the user, the light receiving device measures a volumetric variation caused by a blood pressure pulse of the skin of the user thereby generating the digitized photoplethysmogram signals to the processing unit, and then a series of heart rate signals in correspondence to the digitized photoplethysmogram signals are transmitted through the heart rate signal transmission device.

2. The system as claimed in claim 1, wherein the grip is adapted to be gripped by a palm of the user.

3. The system as claimed in claim 1, wherein the grip is installed on one of indoor fitness equipment, an outdoor bike and a scooter.

4. The system as claimed in claim 1, wherein the grip is installed on a steering wheel.

5. The system as claimed in claim 1, wherein the heart rate signal transmission device is electrically connected to a display.

6. The system as claimed in claim 1, wherein the heart rate signal transmission device is a wireless heart rate signal transmission device, which further wirelessly transmits the heart rate signals to a portable electronic device.

7. The system as claimed in claim 1, wherein the heart rate signal transmission device is connected to a fitness console of one of an indoor fitness equipment, an outdoor bike and a scooter through a connector to transmit the heart rate signals to the fitness console, and the fitness console supplies the operating voltage to the photoplethysmogram signal detector through the connector.

8. The system as claimed in claim 1, further comprising a motion sensor electrically connected to the processing unit for generating a motion detection signal to the processing unit.

9. The system as claimed in claim 7, wherein the motion sensor is selected from one of vibration detector and accelerometer.

10. The system as claimed in claim 1, wherein the detection actuating unit is a capacitive detection actuating unit disposed on the carrier of the photoplethysmogram signal detector.

11. The system as claimed in claim 1, wherein the detection actuating unit is a resistive detection actuating unit disposed on the carrier of the photoplethysmogram signal detector.

12. The system as claimed in claim 1, wherein the power supply device is built in the photoplethysmogram signal detector.

13. The system as claimed in claim 1, wherein the power supply device is an external power supply device which supplies the operating voltage to the photoplethysmogram signal detector.

14. The system as claimed in claim 1, wherein the optical grating further comprises a surrounding portion which surrounds the light emitting device and the light receiving device.
